(19) United States
(12) Patent Application Publication
(10) Pub. No.: US 2021/0386277 A1
(43) Pub. Date: Dec. 16, 2021

(54) METHODS OF CHARACTERISING AND IMAGING WITH AN OPTICAL SYSTEM

(71) Applicant: Cancer Research Technology Limited, London (GB)

(72) Inventors: George Sinclair Dukoff GORDON, London (GB); Calum WILLIAMS, London (GB); Sarah Elizabeth BOHNDIEK, London (GB)

(21) Appl. No.: 17/291,025

(22) PCT Filed: Nov. 11, 2019
(86) PCT No.: PCT/GB2019/053195

§ 371 Date: May 4, 2021

(30) Foreign Application Priority Data
Nov. 9, 2018 (GB) 1818290.7

(51) Int. Cl. A61B 1/00; A61B 5/00; G02B 6/42

(52) U.S. Cl. CPC A61B 1/00165; A61B 1/00057; G02B 6/4215; A61B 5/4887; A61B 5/0084; A61B 1/00096

(57) ABSTRACT

A method of characterizing an optical system, wherein the optical system comprises an optical fibre having a proximal end and a distal end, wherein the optical fibre comprises a non-single-mode optical fibre, and comprising a reflector assembly comprising a stack of reflectors disposed at the distal end of the optical fibre, wherein the stack of reflectors is arranged to provide different reflector matrices in dependence on illumination wavelength. The method comprises projecting calibration patterns at a plurality of characterization wavelengths onto the proximal end, then obtaining, at the proximal end, data relating to reflected calibration patterns. An instantaneous transmission matrix of the optical fibre is then determined using the data relating to the reflected calibration patterns and the reflector matrices.

This present disclosure relates to a method of characterizing an optical system, particularly, but not exclusively where the optical system is used as an imaging system. Aspects of the invention relate to a method of imaging, to an optical system, and to an imaging system that includes the optical system.

### BACKGROUND

Imaging through optical fibres is known and is becoming increasingly common. White light imaging through certain types of optical fibres, termed imaging fibre bundles or multicore fibre (MCF), is a well-established technique used in commercial boresocopes and medical endoscopes. More recently, coherent light (i.e. light from a laser) has been used to image through other kinds of fibre, such as multimode fibre (MMF). The advantage of using coherent light is that images can be formed without lenses, meaning endoscopes need not be any thicker than the fibre itself (often <500 μm). In the case of MMF imaging, this size advantage is enhanced due to the higher ‘information density’—that is, MMF provides more pixels of resolution per unit area than MCF. This opens up opportunities for minimally invasive optical imaging in previously inaccessible areas of the body, e.g. deep in the brain. As a result, there have been a number of studies examining the use of both MMF and MCF for coherent light imaging.

White-light endoscopy is the standard-of-care for inspecting large areas of the gastrointestinal (GI) tract and lung for pre-malignant change (dysplasia) and cancer. For example, Barrett's oesophagus is an acquired metaplastic condition that predisposes patients to the development of oesophageal adenocarcinoma. The cancer risk for Barrett's patients increases significantly in the presence of dysplasia, up to more than 30% per year. Early identification of dysplasia enables curative intervention through simple endoscopic resection or radiofrequency ablation. Unfortunately, the current surveillance procedure uses white-light endoscopy combined with random biopsy, which together show only a 40-64% sensitivity for dysplasia, leading to high miss rates. The 5-year survival rate for oesophageal cancer is only 15%, yet can be as high as 80% when patients are diagnosed with early-stage disease, hence improvements in endoscopic early detection methodologies are urgently needed. While application of dyes can improve contrast, their use lengthens procedure times and can lead to toxicities. Label-free approaches could better address the clinical unmet need for improved contrast of dyplastic tissue.

Localised proliferation of cells in dysplasia scatters light, creating abnormally distorted wavefronts. Phase imaging has been shown to be highly sensitive to such distortions. Furthermore, polarimetric imaging measurements of diattenuation, retardance and circularity can be modified by scattering, as well as perturbed by the higher concentrations of optically anisotropic molecules such as collagen abundant in tumours. Light scattering spectroscopy has shown promise for detecting dysplasia using phase and polarisation information, but interrogates only a narrow field-of-view. This limitation is partially mitigated by optical coherence tomography, which uses lateral scanning mechanisms, but interpretation of the resulting cross-sectional images remains challenging. In addition, both approaches require dedicated complex instrumentation, which has limited endoscopic application.

Flexible medical fibrescopes relay optical information from within the patient to the imaging system outside, which could be used to enable direct, wide-field, phase and polarisation imaging in existing clinically approved systems with comparatively simple and low-cost elements, such as coded apertures, gratings, and polarising optics. Commercial endoscopes typically use distal sensors (‘chip-on-tip’) and although prototype devices with distal optics for other modalities have been developed (e.g. holographic imaging) the additional bulk (>2-fold width) makes integration with existing endoscopic procedures difficult.

Fibre bundles are typically <1 mm in width, independent of the imaging modality, making them attractive candidates for implementation of novel medical imaging technologies in endoscopy. However, both MCF and MMF scatter light in a deterministic but highly complex manner that is a function of bending and temperature. This scattering prevents imaging in most cases, or at the very least greatly reduces imaging quality, and so must be counteracted. This can be achieved with very high accuracy using transmission matrix (TM) approaches to pre-characterise the full optical transfer properties of the fibre before imaging. Typically, the TM is measured by sending known light fields in one end of the fibre and measuring what comes out at the other. By creating many different vectors x₁=[x₁ . . . x_{M}]^{T} and measuring the corresponding output field y₁=[y₁ . . . y_{N}]^{T}, a linear system may be built up:

Y=AX

where
Y=[y₁ y₂ . . . y_{P}]
X=[x₁ x₂ . . . x_{P}]

These equations can then be solved to determine A, the transmission matrix (TM), for example by computing:

YX⁻¹=A

An actual image vector, xₛ, will be transformed by the fibre to produce a raw output, yₛ=Axₛ. To recover xₛ from measured yₛ, the following equation is used: xₛ=A⁻¹yₛ.

However, there remains a key limitation of most work to date using TM characterisation for fibre imaging. This is that known light fields must be sent in one facet fibre and measured at the other in order to obtain the TM. This poses a problem for realistic use because one end of the fibre (the distal facet) is likely to be inaccessible, for example deep inside the body. It is undesirable to introduce a scanning mechanism at the distal facet as this would introduce additional bulk thereby reducing a key benefit of the technique: that the imaging fibre is hair thin (<500 μm). Although miniaturised scanning mechanisms have been integrated into endoscopes of diameter 1 mm, such approaches are not easily scalable to allow additional advanced processing, e.g. polarimetry or phase detection. Having all optics located at the proximal facet outside the body allows much more flexibility for advanced imaging. Such a system with no distal optics cannot simply be achieved by premeasuring the fibre TM because the fibre is extremely sensitive to minor changes in bending and temperature as would be encountered in vivo. A pre-measured TM is therefore not sufficient for image recovery in practical applications.

It is an object of embodiments of the present invention to overcome certain disadvantages associated with the prior art.

### BRIEF SUMMARY OF THE DISCLOSURE

In accordance with an aspect of the present invention, there is provided a method of characterizing an optical system, wherein the optical system comprises:
an optical fibre having a proximal end and a distal end; and
a reflector assembly comprising a stack of reflectors disposed at the distal end of the optical fibre, wherein the stack of reflectors is arranged to provide different reflector matrices in dependence on illumination wavelength;

the method comprising:
projecting calibration patterns at a plurality of characterization wavelengths onto the proximal end;
obtaining, at the proximal end, data relating to reflected calibration patterns; and
determining an instantaneous transmission matrix of the optical fibre using the data relating to the reflected calibration patterns and the reflector matrices.

In certain embodiments, the reflector matrices may be determined by:
determining a characterization transmission matrix of the optical fibre in a characterization configuration;
determining, at each of the plurality of characterization wavelengths, a reflectance matrix of the optical fibre in the characterization configuration with the stack of reflectors disposed at the distal end; and
determining the reflector matrices using the reflectance matrices and the characterization transmission matrix.

Determining the characterization transmission matrix may comprise transmitting light through the optical fibre in the characterization configuration at each of the plurality of characterization wavelengths, detecting the transmitted light at each of the plurality of characterization wavelengths, determining the characterization transmission matrix using the detected transmitted light.

Detecting reflected calibration patterns may comprise measuring the amplitude of reflected calibration patterns. Additionally or alternatively, detecting reflected calibration patterns comprises measuring the phase of reflected calibration patterns. Additionally or alternatively, detecting reflected calibration patterns comprises measuring the polarisation of reflected calibration patterns.

The method may comprise producing a square reflectance matrix from the reflected calibration patterns, wherein determining the instantaneous transmission matrix comprises using the square reflectance matrix.

In accordance with another aspect of the present invention, there is provided a method of imaging comprising:
providing an optical system comprising an optical fibre having a proximal end and a distal end, and a reflector assembly comprising a stack of reflectors disposed at the distal end of the optical fibre, wherein the stack of reflectors is arranged to provide different reflector matrices in dependence on illumination wavelength;
characterizing the optical system as described above;
illuminating a sample proximate to the distal end at an imaging wavelength selected from the characterization wavelengths;
obtaining, at the proximal end, image data relating to the sample; and
producing recovered image data using the image data relating to the sample and the instantaneous transmission matrix.

The method may further comprise obtaining a transmission matrix of the reflector assembly, wherein determining the instantaneous transmission matrix or producing recovered image data may comprise using the transmission matrix of the reflector assembly.

Producing recovered image data may comprise producing recovered amplitude data. Additionally or alternatively, producing recovered image data may comprise producing recovered phase data. Additionally or alternatively, producing recovered image data may comprise producing recovered polarisation data. In certain non-limiting embodiments, the sample may comprise human or animal tissue. The sample may be in vivo, ex vivo or in vitro.

In accordance with another aspect of the present invention, there is provided an optical system comprising an optical fibre having a proximal end and a distal end, and a reflector assembly comprising a stack of reflectors disposed at the distal end of the optical fibre, wherein stack of reflectors is arranged to provide different reflector matrices in dependence on illumination wavelength.

The stack of reflectors may comprise a plurality of reflectors. Each of the plurality of reflectors may be separated from an adjacent reflector by an absorptive filter. The plurality of reflectors may comprise optical metasurfaces.

In certain embodiments, the optical system may further comprise:
an illumination source optically coupled to the optical fibre;
means for generating a calibration pattern for projection onto the proximal end of the optical fibre; and
detection means for detecting images from the proximal end of the optical fibre.

In accordance with another aspect of the present invention, there is provided a method of determining a presence of a physiological condition in a subject, the method comprising:
producing recovered image data relating to a tissue sample using a method as described above or the optical system described above; and
determining, in dependence on the recovered image data, a presence of the physiological condition in the tissue sample.

The step of determining a presence of the physiological condition in the tissue sample may comprise:

using the recovered image data as contrast mechanisms to identify tissue morphology or microstructure associated with the physiological condition; and/or

using the recovered image data to produce images showing the spatial variation or spatial entropy of one or more of amplitude, phase and polarisation, and using differences thereof to delineate healthy areas of the tissue sample (e.g. areas not affected by the physiological condition) and areas of the tissue sample that are affected by the physiological condition (e.g. as described below with reference to FIG. 11).

The physiological condition may, for example, be cancer or a pre-cancerous condition, the presence of scar tissue, or the presence of inflammation. The above described method may be useful in determining the presence of other physiological conditions.

In accordance with another aspect of the present invention, there is provided a computer implemented method comprising:
receiving recovered image data relating to a tissue sample obtained using a method as described above or the optical system described above; and
determining, in dependence on the recovered image data, a presence of a physiological condition in the tissue sample.

The step of determining a presence of the physiological condition in the tissue sample may comprise:

using the recovered image data as contrast mechanisms to identify tissue morphology or microstructure associated with the physiological condition; and/or
using the recovered image data to produce images showing the spatial variation or spatial entropy of one or more of amplitude, phase and polarisation, and using differences thereof to delineate healthy areas of the tissue sample (e.g. areas not affected by the physiological condition) and areas of the tissue sample that are affected by the physiological condition (e.g. as described above with reference to FIG. 11).

The physiological condition may, for example, be cancer or a pre-cancerous condition, the presence of scar tissue, or the presence of inflammation. The above described method may be useful in determining the presence of other physiological conditions.

### BRIEF DESCRIPTION OF THE DRAWINGS

Embodiments of the invention are further described hereinafter with reference to the accompanying drawings, in which:

FIG. 1A is a schematic view of an optical system according to an embodiment of the present invention;

FIG. 1B is a schematic view of an optical system according to an alternative embodiment of the present invention;

FIG. 2A is a schematic view of a part of the optical system of FIGS. 1A and 1B in a transmission mode with the optical fibre in a characterization configuration;

FIG. 2B is a schematic view of a part of the optical system of FIGS. 1A and 1B in a reflection mode with the optical fibre in the characterization configuration;

FIG. 2C is a schematic view of a part of the optical system of FIGS. 1A and 1B in a transmission mode with the optical fibre in a characterization configuration;

FIG. 2D shows a schematic physical model used for fibre TM characterisation in reflection mode in accordance with embodiments of the present invention;

FIG. 2E shows a schematic physical model used for fibre imaging in accordance with embodiments of the present invention;

FIG. 3A is a schematic view of an optical fibre and a 3-layer reflector assembly in accordance with an embodiment of the present invention;

FIG. 3B is a graph showing the transmission characteristics of the reflector assembly of FIG. 3A;

FIG. 4A is a schematic view of an optical fibre and a 4-layer reflector assembly in accordance with an embodiment of the present invention

FIG. 4B is a graph showing the transmission characteristics of the reflector assembly of FIG. 4A;

FIG. 5A is a schematic view of a reflector in accordance with an embodiment of the present invention;

FIGS. 5B and 5C are electron micrographs of reflectors in accordance with embodiments of the present invention;

FIG. 6 illustrates a method of determining reflector matrices according to an embodiment of the present invention;

FIG. 7 illustrates a method of determining an instantaneous transmission matrix according to an embodiment of the present invention;

FIG. 8 illustrates a non-limiting detailed example of the steps of projecting calibration patterns and detecting reflected calibration patterns from the method of FIG. 7;

FIG. 9 illustrates a method of imaging according to an embodiment of the present invention;

FIG. 10 shows results relating to label-free identification of early lesions in mouse oesophagus, where: part (a) is a composite image showing sections of healthy tissue and lesions from 9 samples and 5 endoscope modalities; part (b) shows the contrast-to-noise ration for the different modalities calculated independently for each of the 6 samples containing lesions; and part (c) shows a receiver operating characteristic curve illustrating performance of different modalities when a binary classifier with varying threshold is applied to discriminate between healthy and lesion tissue;

FIG. 11 shows a method of producing an image of entropy/mean from recovered image data in accordance with an embodiment of the present invention;

FIG. 12A shows an example of original transmission matrices and corresponding first-order recovered transmission matrices in accordance with an embodiment of the present invention;

FIG. 12B shows the proportional element-wise error in the transmission matrix reconstruction of FIG. 12A;

FIG. 13A shows a further example of original transmission matrices and corresponding first-order recovered transmission matrices in accordance with an embodiment of the present invention;

FIG. 13B shows the proportional element-wise error in the transmission matrix reconstruction of FIG. 13A; and

FIG. 14 shows an example of simulated reconstruction of an amplitude, phase and polarisation image of a target using an experimentally measured transmission matrix in accordance with an embodiment of the present invention.

### DETAILED DESCRIPTION

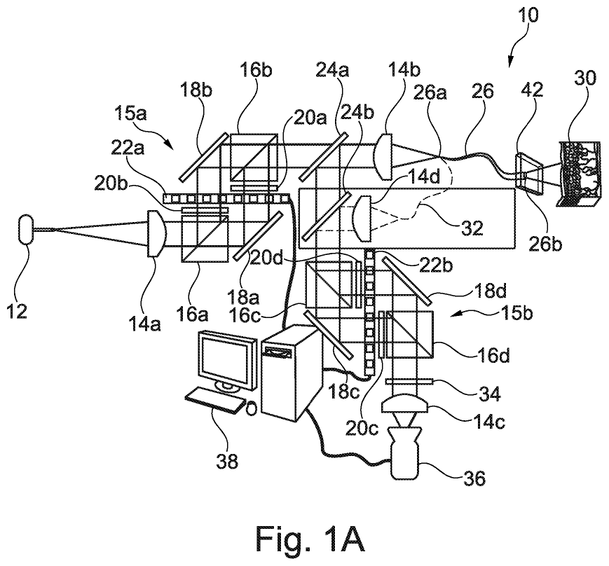

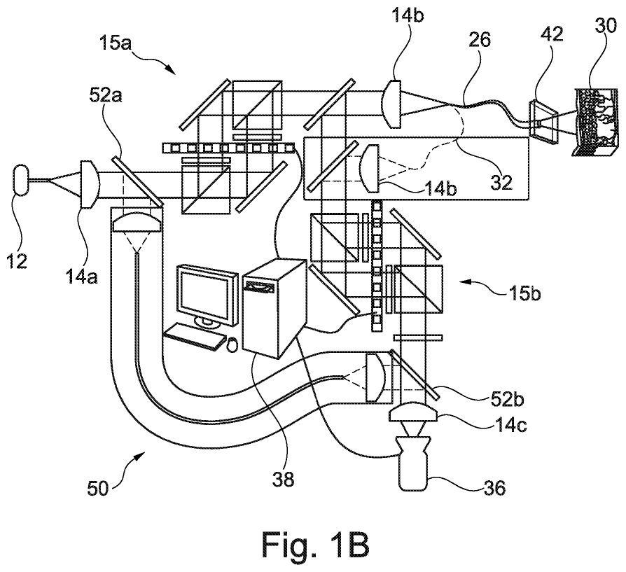

FIG. 1A shows a schematic view of an optical system 10 in accordance with an embodiment of the present invention. The optical system 10 includes a light source 12 which can provide illumination at a plurality of wavelengths. In preferable embodiments, the light source 12 is a tunable laser. A first lens 14a collimates a light beam provided by the light source 12 prior to the light beam being split by a first polarizing beam splitter (PBS) 16a. The transmitted part of the split beam is reflected by a first beam reflector 18a so that it passes through a first spatial light modulator (SLM) 22a (which constitutes means for generating a calibration pattern) prior to passing through a first half wave plate 20a and a second PBS 16b. The reflected part of the beam split by the first PBS 16a passes through a second half wave plate 20b prior to passing through the first SLM 22a and being reflected by a second beam reflector 18b. This part of the beam then passes through the second PBS 16b so as to be combined with the transmitted part of the beam. The combination of the first PBS 16a, second PBS 16b, first half wave plate 20a, second half wave plate 20b, first SLM 22a, first beam reflector 18a and second beam reflector 18b form a first holographic sub-system 15a.

The recombined beam then passes through a first non-polarising beam splitter (NPBS) 24a before being focused by a second lens 14b. The focused beam then passes into an optical fibre 26. The optical fibre 26 may comprise any suitable type of fibre that is capable of facilitating optical propagation. In certain embodiments, the optical fibre 26 may comprise a non-single-mode optical fibre (i.e. an optical fibre that does not behave as a single-mode optical fibre). In particular, the non-single-mode optical fibre may be an imaging fibre bundle or multicore fibre (MCF), a multimode fibre (MMF), dual-clad fibre, photonic crystal fibre, hollow-core fibre, or other microstructured optical fibre. Where such fibres are available in single-mode and multi-mode variants, the term “non-single-mode” in the present application is not intended to encompass to the single-mode variants.

The optical fibre 26 has a proximal end 26a and a distal end 26b. In the configuration shown in FIG. 1A, the proximal end 26a is optically coupled to the light beam focused by the second lens 14b while the distal end 26b is positioned for imaging a sample 30.

The first holographic sub-system 15a described above enables control of optical amplitude, phase and polarization of light entering the proximal end 26a of the optical fibre 26, enabling creation of arbitrary calibration patterns during characterization, and arbitrary illuminations during imaging (described further below).

The sample 30 may be (but is not necessarily) human or animal tissue which may be in vivo, ex vivo or in vitro. The human or animal tissue may form or have formed part of the gastrointestinal tract or respiratory system of a subject.

A reflector assembly 42 (described further below) is disposed on the distal end 26b of the optical fibre 26. Light exiting the distal end 26b of the optical fibre 26 is reflected by the sample, and is returned into the distal end 26b back towards the second lens 14b which then acts to collimate the light beam. The collimated light beam is reflected by the first NPBS 24a so as to separate it from the beam propagating in the opposite direction (i.e. towards the optical fibre 26).

The separated beam passes through a second NPBS 24b before being split by a third PBS 16c. The transmitted part of the split beam is reflected by a third beam reflector 18c so that it passes through a second SLM 22b prior to passing through a third half wave plate 20c and a fourth PBS 16d. The reflected part of the beam split by the third PBS 16c passes through a fourth half wave plate 20d prior to passing through the second SLM 22b and being reflected by a fourth beam reflector 18d. This part of the beam then passes through the fourth PBS 16d so as to be combined with the transmitted part of the beam. The combination of the third PBS 16c, fourth PBS 16d, third half wave plate 20c, fourth half wave plate 20d, second SLM 22b, third beam reflector 18c and fourth beam reflector 18d form a second holographic sub-system 15b.

The combined beam then passes through a 45-degree polarizer 34, is focused by a third lens 14c, and is then detected by detection means in the form of a detector 36. The detector 36 may comprise, for example, a CCD. Second holographic sub-system 15b provides a means of enabling the detector to determine the optical phase and polarisation, in addition to amplitude/intensity of the detected beam.

Prior to first use for imaging, the optical system 10 must be characterized. In particular, the optical fibre 26 must be characterized first, followed by the reflectors 42. As is described below, the characterization of the optical system 10 may utilize an alternative configuration of the optical fibre 26 (path shown by dotted line 32 in FIG. 1A) and a fourth lens 14d.

Processing means in the form of a processor (e.g. within a PC) 38 is communicably coupled to the first SLM 22a for controlling patterns (e.g. holograms) displayed by the first SLM 22a and is similarly coupled to the second SLM 22b. The processor 38 is also communicably coupled to the detector 36 and is configured to receive data obtained by the detector 38.

FIG. 1B shows a schematic view of the optical system 10 of FIG. 1A with an optional interferometer arm 50. In particular, a first interferometer NPBS 52a is positioned between the first lens 14a and the first holographic sub-system 15a. This splits the beam provided by the light source 12 and the interferometer arm 50 directs it to a second NPBS positioned between the 45-degree polarizer 34 and the third lens 14c. Therefore, the interferometer arm 50 provides a reference beam from the light source 12 to the detector 36 so that properties (e.g. the phase) of the original and imaging beams may be compared against one another.

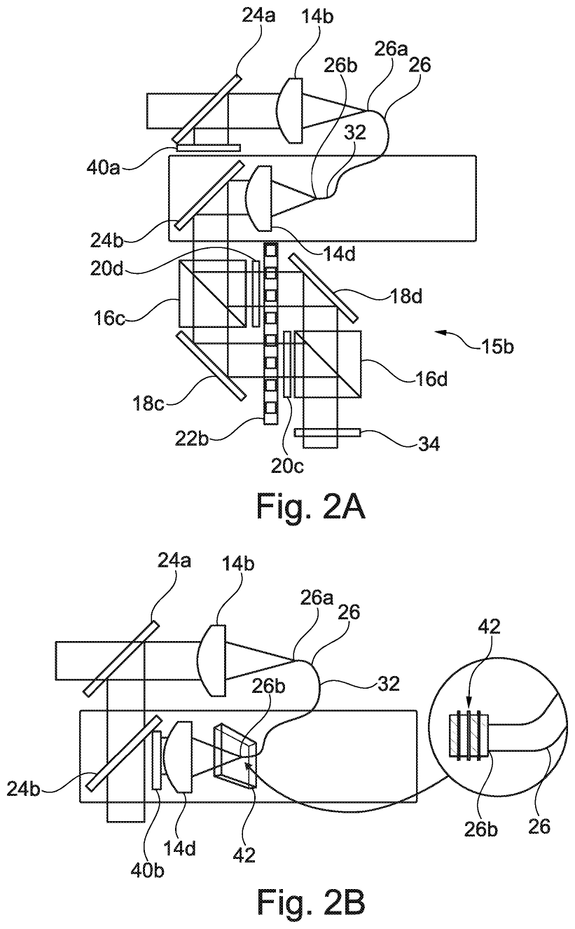

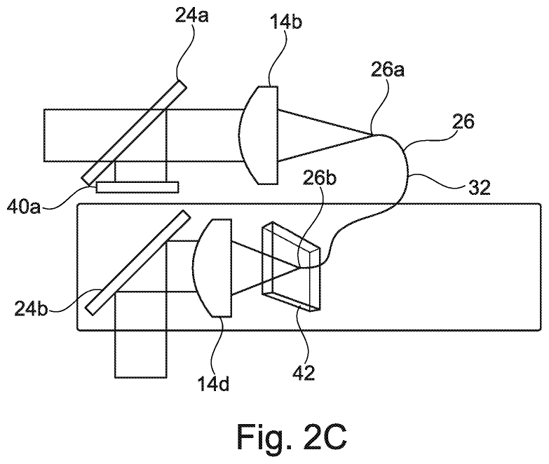

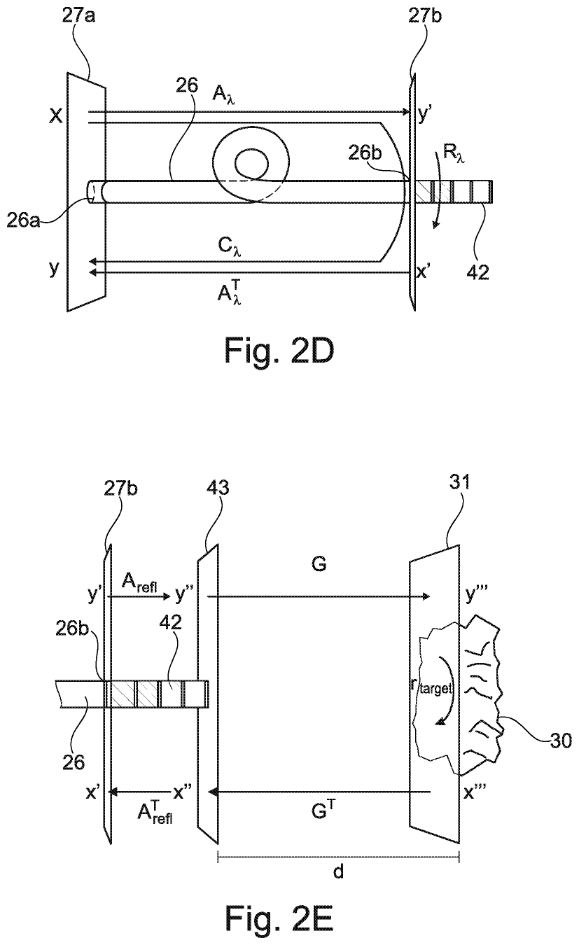

FIG. 2A is a schematic view of a part of the optical systems 10 of FIGS. 1A and 1B in a transmission mode with the optical fibre 26 in a characterization configuration. In particular, the optical fibre 26 follows the path 32 so that light entering proximal end 26a is transmitted along the optical fibre 26 and exits at distal end 26b before being collimated by the fourth lens 14d. The collimated beam then passes to the second holographic sub-system 15b (described above) via the second NPBS 24b before being detected by the detector 36. Meanwhile, a first beam stop 40a prevents light that has not been transmitted through the optical fibre 26 from reaching the second holographic sub-system 15b.

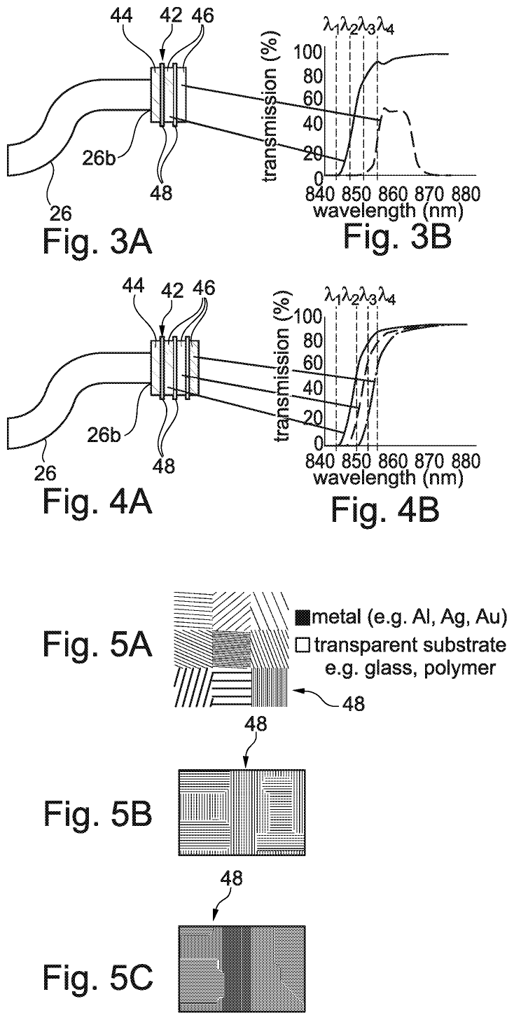

FIG. 2B is a schematic view of a part of the optical systems of FIGS. 1A and 1B in a reflection mode with the optical fibre 26 in the characterization configuration and with the addition of the reflector assembly 42. That is, the optical fibre 26 is in substantially the same physical configuration as it was when the optical system 10 was in the transmission mode (i.e. that described above with reference to FIG. 2A). In contrast to the configuration described above in relation to FIG. 2A, the first beam stop 40a is no longer present and a second beam stop 40b is positioned to prevent light that is exiting the distal end 26b of the optical fibre 26 from reaching the second NPBS 24b (and, thus, the second holographic sub-system 15b). Rather, light that is reflected by the reflector assembly 42 exits the proximal end 26a of the optical fibre 26 and is then collimated by the second lens 14b and directed towards the second holographic sub-system 15b by the first NPBS 24a. After passing through the second holographic sub-system 15b, the light is detected by the detector 36. FIG. 2B additionally shows a detailed view of the reflector assembly 42. Specific embodiments of the reflector assembly 42 are described below with reference to FIGS. 3A, 3B, 4A and 4B.

FIG. 2C is a schematic view of a part of the optical systems of FIGS. 1A and 1B in a transmission mode with the optical fibre 26 in the characterization configuration and with the reflector assembly 42 in place. That is, the optical fibre 26 is in substantially the same physical configuration as it was when the optical system 10 was in the transmission mode (i.e. that described above with reference to FIG. 2A). In particular, the optical fibre 26 follows the path 32 so that light entering proximal end 26a is transmitted along the optical fibre 26, exits at the distal end 26b, and passes through the reflector assembly 42 before being collimated by the fourth lens 14d. The collimated beam then passes to the second holographic sub-system 15b via the second NPBS 24b before being detected by the detector 36. Meanwhile, the first beam stop 40a prevents light that has not been transmitted through the optical fibre 26 from reaching the second holographic sub-system 15b.

FIG. 3A shows an embodiment of the reflector assembly 42 which comprises a “stack of reflectors” formed by a glass layer 44, a pair of reflectors 48 and a pair of absorptive filters 46. In a direction moving out of the distal end 26b of the optical fibre 26, the order of components is: glass layer 44, reflector 48, absorptive filter 46, reflector 48, absorptive filter 46. FIG. 3B shows an example transmission profile for the reflector assembly 42 of FIG. 3A in dependence of wavelength. Whilst the transmission profile shown for the final absorptive filter 46 is indicative of a bandpass filter, the final (or, indeed, any) absorptive filter 46 may be a bandpass or long pass filter. Each of the reflectors 48 may be any surface or structure capable of reflecting light. In certain embodiments, the reflectors 48 may each comprise an optical meta surface. For example, the reflectors 48 may comprise plasmonic reflectors with spatially heterogeneous linear dichroism (or diattenuation). Such structures may be fabricated using electron beam lithography patterning followed by metal deposition to create high-resolution wire-grid polarisers. Suitable methods and examples of such structures are described in Williams et al. (C. Williams, R. Bartholomew, G. Rughoobur, G. S. D. Gordon, A. J. Flewitt, and T. D. Wilkinson, “Fabrication of nanostructured transmissive optical devices on ITO-glass with UV1116 photoresist using high-energy electron beam lithography,” Nanotechnology, vol. 27, no. 48, p. 485301, 2016.) which is hereby incorporated by reference in its entirety, and particularly in respect of the fabrication methods and wire-grid plasmonic polarisers described therein. FIGS. 5A, 5B and 5C show exemplary reflectors 48 in accordance with embodiments of the present invention. The reflectors 48 shown in FIGS. 5A, 5B and 5C are plasmonic reflectors with heterogeneous diattenuation. FIG. 5A highlights the distinction between metallic regions and regions of a transparent substrate. FIG. 5B shows an electron micrograph of a reflector 48 made of silver on a glass substrate. FIG. 5C shows an electron micrograph of a reflector 48 made of aluminium on a glass substrate.

FIG. 4A shows an alternative embodiment of the reflector assembly 42 which comprises a “stack of reflectors” that includes a glass layer 44, three reflectors 48 and three absorptive filters 46. In a direction moving out of the distal end 26b of the optical fibre 26, the order of components is: glass layer 44, reflector 48, absorptive filter 46, reflector 48, absorptive filter 46, reflector 48, absorptive filter 46. FIG. 4B shows an example transmission profile for the reflector assembly 42 of FIG. 4A in dependence of wavelength.

At different wavelengths, the reflector assembly 42 provides a different reflector matrix, effectively switching between reflectors 48. On the assumption that the transmission matrix of the optical fibre 26 is relatively similar at each of these different wavelengths, the transmission matrix may be determined using the reflector matrices, as is described below.

In alternative embodiments, the stack of reflectors may comprise other numbers of reflectors and/or absorptive filters, and/or be any other arrangement that is capable of providing a reflector matrix that is dependent on the wavelength of incident light.

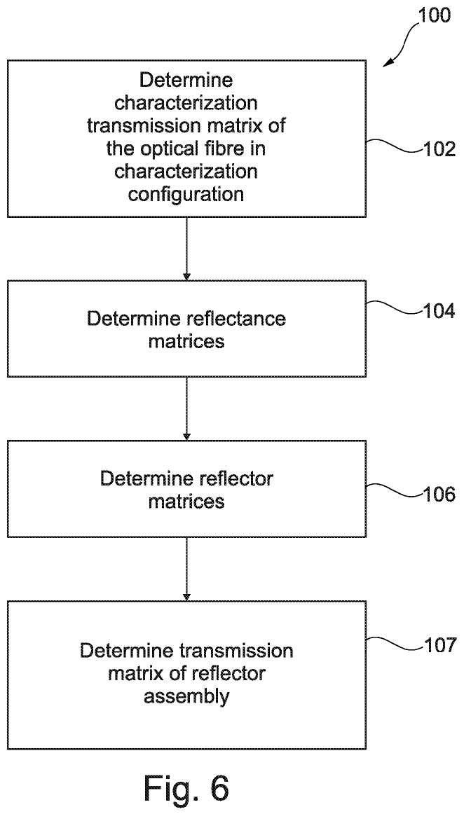

FIG. 6 illustrates a method 100 according to an embodiment of the present invention. The method 100 is a method of determining reflector matrices. In the transmission mode with the optical fibre in the characterization configuration (as shown in FIG. 2A) light from the light source 12 is transmitted through the optical fibre 26 and detected by detector 36. In this manner, a transmission matrix may be measured for each of a plurality of characterisation wavelengths, λ₁, λ₂, λ₃, . . . , λ_{Q}. The measured transmission matrices A₁, A₂, A₃, . . . , A_{Q} are assumed to be very similar to one another over the selected wavelength range and so may be averaged or otherwise combined (for example by interpolation) to determine the “characterization transmission matrix”, A, of the optical fibre 26 (step 102 of method 100).

Next, maintaining the characterization configuration, the reflector assembly 42 is inserted and butt-coupled against the distal end 26b of the optical fibre 26 (as shown in FIG. 2B). In this reflection mode, at step 104, reflectance matrices, M, of the optical fibre 26 and reflector assembly 42 combination are measured at the characterization wavelengths, λ₁, λ₂, λ₃, . . . , λ_{Q}.

Using the determined characterization transmission matrices of the optical fibre 26 and the measured reflectance matrices, the reflector matrices, R, can be determined (step 106) since M₁=A₁^{T}R₁A₁, and R₁=(A₁^{T})⁻¹M₁A₁⁻¹.

Next, the second beam stop 40b is removed and the first beam stop 40a is replaced (to adopt the configuration shown in FIG. 2C) to enable measurement of the transmission matrix, Z, of the reflector assembly 42. Using the configuration of FIG. 2C, light is transmitted through the optical fibre 26 and reflector assembly 42, with the transmitted beam passing to the second holographic sub-system 15b. This set up allows the transmission matrix, P, of the combination of the optical fibre 26 and reflector assembly 42 to be measured. Using the relationship Zᵢₘ=PᵢₘA_{Q+1}⁻¹, where Pᵢₘ is the transmission matrix of the combination of the optical fibre 26 and reflector assembly 42 at the imaging wavelength (λ_{Q+1}), and A_{Q+1} is the transmission matrix of the optical fibre 26 at the imaging wavelength, the transmission matrix, Zᵢₘ, of the reflector assembly 42 at the imaging wavelength may be determined (step 107).

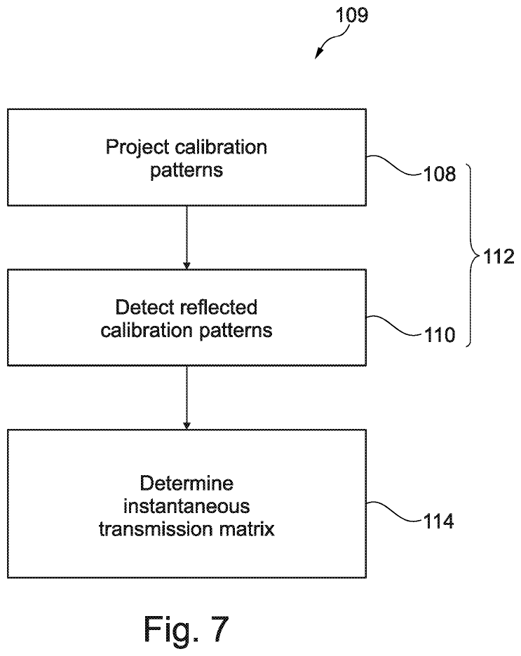

Prior to acquisition of sample image data, some further characterization is required (this time in the imaging configuration as shown in FIGS. 1A and 1B). FIG. 7 illustrates a method 109 of determining an instantaneous transmission matrix (i.e. the transmission matrix corresponding to the instantaneous conditions, e.g. configuration or temperature, when in the imaging configuration) according to an embodiment of the present invention. The method 109 comprises, at step 108, projecting calibration patterns at a plurality of characterization wavelengths onto the proximal end 26a of the optical fibre 26. The data relating to reflected calibration patterns is obtained at the proximal end 26a at step 110, and the instantaneous transmission matrix of the optical fibre 26 is determined at step 114 using the data relating to the reflected calibration patterns and the reflector matrices determined in step 106.

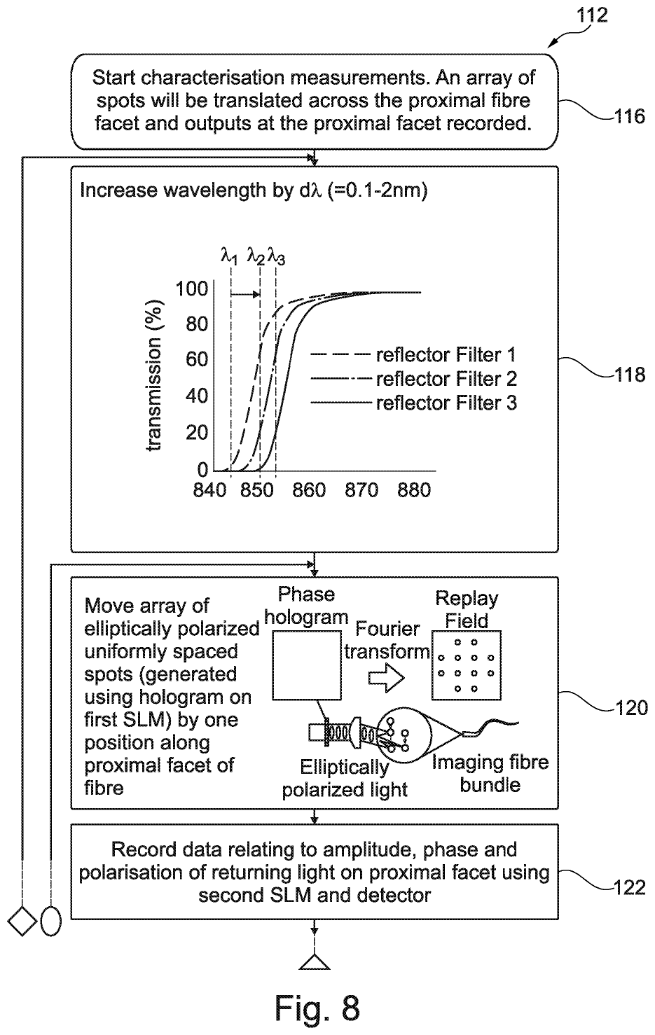

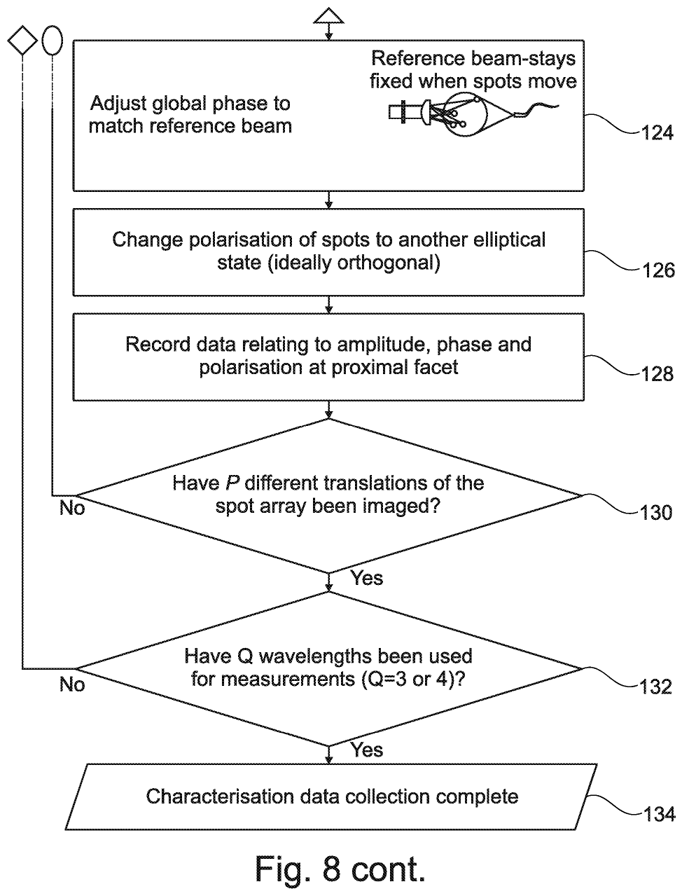

FIG. 8 illustrates a detailed non-limiting example of step 112 (which is a compound of steps 108 and 110) from the method 109, for projecting calibration patterns and measuring reflected calibration patterns. Characterization measurements are started at step 116. A first characterization wavelength is selected at step 118 and the light source 12 produces an array of spots at the characterization wavelength which is then projected onto the proximal end (“facet”) 26a of the optical fibre 26. The array may comprise an array of elliptically polarized, uniformly spaced spots. The array is generated using a hologram on the first SLM 22a. At step 120, the array is moved by one position along the proximal facet 26a of the optical fibre 26. At step 122, data relating to the amplitude, phase and polarization of light returning (i.e. being reflected) to the proximal facet 26a of the optical fibre 26 is measured using the second SLM 22b and the detector 36. Next, at step 124, the hologram on the first SLM 22a is adjusted so as to continue to project the array of spots onto the proximal facet 26a, but so that it simultaneously displays an additional spot (“reference beam”) at a fixed position on the proximal facet 26a that does not change as the array of spots is moved. In step 124, the phase of the reference beam is measured to determine the required global phase needed for instantaneous transmission matrix estimation. At step 126, the polarization of the array of spots is changed to another elliptical state (e.g. orthogonal to the previous state). Data relating to the amplitude, phase and polarization of the light at the proximal facet 26a is measured by the second SLM 22b and detector 36 at step 128. Step 130 checks if a sufficient number, P, of translations of the array of spots have been imaged (i.e. measured by the detector 36). If no, then steps 120 to 128 are repeated before the check at step 130 is repeated. If yes, then step 132 checks if a sufficient number, Q, of wavelengths have been used for the measurements (e.g. Q=3 or 4, depending on the reflector assembly used). If no, a new characterization wavelength is selected at step 118 and the light source 12 produces an array of spots at the new characterization wavelength which is then projected onto the proximal end (“facet”) 26a of the optical fibre 26. The new characterization wavelength may differ from the previous characterization wavelength by an amount dλ, which may, for example, be between 0.1 to 2 nm. Using the new characterization wavelength, steps 120 to 130 are repeated before the check at step 132 is repeated. Once the sufficient number, Q, of wavelengths have been used for the measurements, the collection of characterization data is complete (step 134).

A physical model used for aiding understanding of certain aspects of the present invention is shown in FIG. 2D. A model is considered where an optical field containing M pixels, each a complex-valued vector encoding amplitude and phase in two orthogonal polarisations, is recorded. The sampled field at some input plane 27a (e.g. the proximal facet 26a), is ordered into a vector x∈ [symbol] ^{2M}, and the sampled field at an output plane 27b (e.g. the distal facet 26b) is similarly ordered into a vector y′∈ [symbol] ^{2M}. In the forward propagation direction, these vectors are related by the monochromatic fibre TM at some wavelength, λ, A_{λ}∈ [symbol] ^{2M×2M}: y′=A_{λ}x. Similarly, considering a field, x′∈ [symbol] ^{2M}, at the distal facet propagating in the reverse direction to become a field, y∈ [symbol] ^{2M}, at the proximal facet 26a, these are related by the transpose of the fibre TM: y=A_{λ}^{T}x′. The impact of adding a reflector assembly 42 at the distal facet 26b of the fibre 26 is then considered. In general, the reflector assembly 42 is considered to be spatially heterogeneous in terms of its localised Jones reflection matrices: there may be uncorrelated Jones matrices describing reflections at each spatial point. Further, if the reflector assembly 42 is offset from the fibre 26, light may couple between spatial positions due to diffraction. This behaviour is linear and so is represented by a partial reflector matrix (PM) at wavelength λ, R_{λ}∈ [symbol] ^{2M×2M}, that relates y′ and x′: x′=R_{λ}y′ (see FIG. 2D). Next, combining the above expressions for y′, y and x′, the optical field exiting the proximal facet 26a in the reverse direction can be determined:

y=A_{λ}^{T}R_{λ}A_{λ}x= [symbol] (1)

where

C_{λ}=A_{λ}^{T}R_{λ}A_{λ}∈ [symbol] ^{2M×2M} (2)

is a reflection matrix (RM). Physically speaking, C_{λ} represents light taking a complete round-trip (or double-pass): forwards down the fibre 26, off a given reflector 48 of the reflector assembly 42 and back up the fibre 26 (as shown in FIG. 2D). Experimentally, it is determined through multiple measurements of vector pairs, (x, y), at the proximal facet 26a of the fibre 26. This may lead to a non-square approximation to C_{λ}, denoted as \tilde{C}_{λ}. To enable computation of matrix exponentials, \tilde{C}_{λ} first needs to be downsampled into a square matrix \ddot{\ddot{C}}₂, which is then used as a surrogate for \tilde{C}_{λ} in what follows. Given that R_{λ} is characterized in advance and it will remain fixed throughout use, the goal is then to recover A_{λ} based on measurements of C_{λ} and R_{λ}. At a single wavelength, λ, this is not in general possible unless the fibre TM is unitary. It is therefore proposed to use several different reflectors 48 with PMs R_{λ} at wavelengths λ=λ_{1 . . .} λ_{Q} and measured RMs C_{λ}, Cλ, λ_{1 . . .} λ_{Q} to enable unambiguous recovery of A_{λ} for any λ=λ_{1 . . .} λ_{Q} in the more general non-unitary case. In order to recover A_{λ} for any λ=λ_{1 . . .} λ_{Q}, given R_{λ}_{_{q}}, C_{λ}_{_{q}} with q=1 . . . Q and Equation (2), the relationship between TMs at different wavelengths must be modelled. Two simple models are considered in turn: a ‘zeroth-order model’ and a ‘first-order model’.

The zeroth-order model assumes that the TMs under the different reflectors 48 are the same, i.e.: A=A_{λ}_{₁}=A_{λ}_{₂}= . . . =A_{λ}_{_{Q}}.

Physically, this corresponds to wavelength modulations significantly less than the spectral bandwidth of the fibre 26. This model has the significant advantage that if at least 3 wavelengths are used (Q≥3) producing at least 3 RMs, A can be recovered in a relatively straightforward way relying largely on analytical steps (described further below). This analytical approach further requires that the eigenvalues of each PM, R_{λ}, λ=λ₁ . . . λ_{Q}, must be distinct for unambiguous TM recovery, in agreement with previous findings. Light must therefore be coupled between different fibre modes and polarisations, so a conventional partial mirror reflector would not work because it preserves polarisation states leading to repeated matrix eigenvalues. This insight informs reflector assembly 42 design. In reality, the relatively small spectral bandwidths of typical imaging fibres (˜5 nm) would require the use of optical components (e.g. filters) with impractically sharp spectral responses to achieve different reflectance behaviour over such a small wavelength range.

By way of further explanation in relation to the zeroth-order model, using the same sampling matrix, T, “distilled” square versions of all two (or three) refection mode matrices can be produced (taken at different wavelengths), which are denoted C₁, C₂, and C₃. Since each of these uses a different reflector 48 but has approximately the same transmission matrix, the following can be written:

C₁=A^{T}R₁A (3)

C₂=A^{T}R₂A (4)

C₃=A^{T}R₃A (5)

The following non-limiting steps may then be taken to recover the instantaneous transmission matrix, A (however any suitable alternative method may be employed).

Starting from (3), (4) and (5):

C₂⁻¹=A⁻¹R₂⁻¹(A^{T})⁻¹

C₂⁻¹C₁=A⁻¹R₂⁻¹(A^{T})⁻¹A^{T}R₁A

C₂⁻¹C₁=A⁻¹R₂⁻¹R₁A

C_{α}=A⁻¹R_{α}A (6)

AC_{α}=R_{α}A

R_{α}A−AC_{α}=0 (7)

where C_{α}=C₂⁻¹C₁ and R_{α}=R₂⁻¹R₁. It is important to note from (6) that C_{α} and R_{α} are similar matrices and so have the same eigenvalues.

In a similar fashion, the following can be derived:

C₃⁻¹=A⁻¹R₃⁻¹(A^{T})⁻¹

C₃⁻¹C₂=A⁻¹R₃⁻¹(A^{T})⁻¹A^{T}R₂A

C₃⁻¹C₂=A⁻¹R₃⁻¹R₂A

C_{β}=A⁻¹R_{β}A (8)

AC_{β}=R_{β}A

R_{β}A−AC_{β}=0 (9)

where C_{β}=C₃⁻¹C₂ and R_{β}=R₃⁻¹R₂.

Both (7) and (9) are examples of Sylvester equations, so known methods of solving them can be employed.

Because (7) and (9) have RHS=0, the trivial solution A=0 satisfies both equations. However, there must exist at least one other non-trivial solution for A. Therefore, an attempt may first be made to determine the space of the non-trivial solutions so that the true answer may be searched for within that space. This may be achieved by implementing a modified version of the Bartels-Stewart algorithm for solving Sylvester equations which is described in R. Bartels and G. W. Stewart (R. Bartels and G. W. Stewart, “Algorithm 432: Solution of the Matrix Equation (AX+XB=C),” Comm. ACM, vol. 15, no. 9, pp. 820-826, 1972.) which is hereby incorporated by reference in its entirety. The advantage of using this approach over other methods (e.g. using Kronecker products to solve for a vectorized matrix, vec(A)) is that its computational complexity is significantly reduced (O(n³) vs. O(n⁶)) as its memory usage.

Using the Schur matrix decomposition (described in R. A. Horn and C. R. Johnson, Matrix analysis. 2013, which is hereby incorporated by reference in its entirety), any square matrix, A, can be decomposed as:

A=QUQ⁻¹ (10)

where Q is unitary and U is an upper triangular matrix. Importantly, the diagonal of U comprises the eigenvalues of A. These are in an arbitrary order, but there are algorithms that enable these to be sorted, e.g. in descending order of magnitude.

From (7) and (10), it can be written:

R_{α}=Q_{R}U_{R}Q_{R}⁻¹

C_{α}=Q_{C}U_{C}Q_{C}⁻¹ (12)

Substituting (11) and (12) back into (7) gives:

Q_{R}U_{R}Q_{R}⁻¹A−AQ_{C}U_{C}Q_{C}⁻¹=0

Q_{R}U_{R}Q_{R}⁻¹AQ_{C}−AQ_{C}U_{C}=0

U_{R}Q_{R}⁻¹AQ_{C}−Q_{R}⁻¹AQ_{C}U_{C}=0

If:

A′=Q_{R}⁻¹AQ_{C} (13)

then:

U_{R}A′−A′U_{C}=0 (14)

(14) is another Sylvester equation but, crucially, U_{R} and U_{C} are upper triangular so it can be solved element by element. If all matrices are square and of size N×N, the element (N, 1) of the RHS zero matrix may be solved first. It can be seen that:

ur_{N,N}a_{N,1}−a_{N,1}uc_{1,1}=0 (15)

where ur_{m,n} is the element in row m, column n of U_{R}, uc_{m,n} is the element in row m, column n of U_{C} and a_{m,n} is the element in row m, column n of A′.

It may be assumed that the eigenvalues of R_{α}, are distinct. This can be arranged by suitable design of the reflectors R₁ and R₂. In general, typical non-unitary reflectors will likely have distinct eigenvalues when characterised experimentally.

Assuming that the eigenvalues of R_{α}, are distinct, it is known that they are equal to the eigenvalues of C_{α}, because the two are similar matrices by (6). Therefore, the values on the diagonal of U_{R} are distinct from one another, and are equal to the values on the diagonal of U_{C}. Therefore, the only way that (15) can hold is if a_{N,1}=0.

Next, element (N−1, 1) is considered, giving:

ur_{N-1,N-1}a_{N-1,1}+ur_{N-1,N}a_{N,1}−a_{N-1,1}uc_{1,1}=0

Since it is known that a_{N,1}=0, by the above reasoning, it is concluded that a_{N-1,1}=0. Continuing on up this column, it is found that all elements must be zero until the first row is reached, where:

ur_{1,1}a_{1,1}−a_{1,1}uc_{1,1}=0

It is known that ur_{m,m}=uc_{m,m} for every m because R_{α}, and C_{α}, are similar matrices by (6). Therefore, any value of a_{1,1} satisfies this equation meaning this element cannot be determined. It is found that diagonal elements, a_{m,m}, are indeterminate but that all other elements in the matrix can be computed from these. For example, the element a_{p,q} with P<Q (i.e. the upper triangular part of A′) is given by:

$$( {ur}_{P , P} - {uc}_{Q , Q} ) {a}_{P , Q} + {\sum }_{q = P + 1}^{Q} {ur}_{P , q} {a}_{q , Q} - {\sum }_{s = P}^{Q - 1} {uc}_{s , Q} {a}_{P , s} = 0$$

Rearranging, we can write:

$$[ - {\sum }_{s = P}^{Q - 1} {uc}_{s , Q} {a}_{P , s} ] + ( {ur}_{P , P} - {uc}_{Q , Q} ) {a}_{P , Q} + {\sum }_{q = P + 1}^{Q} {ur}_{P , q} {a}_{q , Q} = 0 \\ [ {ur}_{P , Q} {a}_{Q , Q} - {\sum }_{s = P}^{Q - 1} {uc}_{s , Q} {a}_{P , s} ] + ( {ur}_{P , P} - {uc}_{Q , Q} ) {a}_{P , Q} + {\sum }_{q = P + 1}^{Q - 1} {ur}_{P , q} {a}_{q , Q} = 0 \qquad ( 16 )$$

If P is varied from 1 to Q−1 in (16), this produces Q−1 equations. If the diagonal elements of A′ are arbitrarily fixed, Q−1 unknowns are left. This system of linear equations can be solved to find all other elements of the P^{th} column of A′. By varying Q and solving each resultant system of equations, all non-diagonal elements of A′ can be fully determined given a set of arbitrary diagonal elements, a_{m,m} (m=1 . . . N). Since every candidate solution of (14) is a linear function of N complex numbers (i.e. of an N-dimensional vector), it can be inferred that the true matrix, A′, can be expressed as a linear combination of at most N matrices, each of which is a solution of (14).

N orthogonal 1×N complex vectors are generated, and, for each of these, the associated solution of (14), termed A′N, is found. Equation (13) is then used to get the associated matrix A_{N} that is a solution to (7). The problem is then reduced to finding a 1×N vector of weights, w=[w₁w₂ . . . w_{N}], such that:

w₁A₁+ . . . +w_{N}A_{N}=A (17)

In effect, the task is to now solve an N-dimensional problem to find an N×N matrix, which is a significant reduction in complexity. A further reduction of the problem is achieved by noting that (17) is an over-determined problem. That is, to uniquely determine the optimal set of weights, w, it is sufficient to consider only a subset comprising the same B (≥N) elements from every A_{N}. This reduced problem of optimizing N weights across B elements is referred to as the weight-reduced solution space.

An alternative reduced solution space can be constructed as follows. If the B relevant element from each A_{N} are taken and put into column vectors b₁ . . . b_{N}, a matrix B can be formed:

B=[b₁ . . . b_{N}] (18)

so that we can write:

Bw^{T}=bₑₛₜ (19)

Where bₑₛₜ is an estimate of the B relevant elements of the true transmission matrix, A. Since B is either square or is a tall matrix, we can pre-multiply by its Moore-Penrose pseudo-inverse, B⁺:

B⁺Bw^{T}=B+bₑₛₜ

Iw^{T}=B⁺bₑₛₜ

w_{T}=B⁺bₑₛₜ

We can then multiply both sides by B to get:

Bw^{T}=BB⁺bₑₛₜ

and substitute in (19):

bₑₜ=BB⁺bₑₛₜ (20)

Clearly, the solution bₑₛₜ must then be an eigenvector of the B×B matrix, B⁺B, and so we call this eigenspace.

If a 4-layer stack design of the reflector assembly 42 (as shown in FIG. 4A) is used, then the reflector assembly 42 of FIG. 4A has three reflectors 48 available so eigenspaces from both C_{α} and C_{β} ((7) and (13) respectively) can be determined. Deriving (20) with some arbitrary amount of reflection from each of the 3 reflectors 48, C_{α} can be obtained, and subsequently B_{α} to get:

bₑₛₜ=B_{α}B_{α}⁺bₑₛₜ (21)

A different amount of reflection from 3 reflectors 48 will produce C_{β} and subsequently B_{β}, and we can re-derive (20) as:

bₑₛₜ=B_{β}B_{β}⁺bₑₛₜ (22)

The different amount of reflection from the three reflectors 48 is determined by the characterisation wavelengths used and the spectral profile of the absorptive filters 46. In certain embodiments these wavelengths and spectral profiles may be determined through an optimisation process to produce maximally different R_{α} and R_{β} and hence B_{α} and B_{β}.

In certain embodiments, bₑₛₜ may be recovered from the above equations by one of two approaches (which are described below). In accordance with other embodiments of the invention, alternative suitable approaches may be adopted. A first approach seeks to achieve optimization using a prior. In general, the elements of transmission matrices tend to follow some pattern even under large perturbations in temperature or bending. For example, in an MCF, it is typically found that there is usually high power coupling between adjacent fibre cores and weak coupling between distant cores. In MMF, modes with similar propagation constants (degenerate modes) experience high power coupling and those with very different propagation constants experience lower coupling.

This idea can be formalized as determining a statistical prior distribution of transmission matrices. To determine such a distribution, many experimental transmission matrix realisations could be measured, i.e. various fibres under randomized bending and temperature conditions, and then fit a multivariate distribution to this data. An estimated transmission matrix may be denoted by Aₑₛₜ and the probability density function of this fitted prior distribution may be denoted by f(Aₑₛₜ). As an example, f(A) might be a complex multivariate Gaussian distribution with some covariance between each element of A. In the weight-reduced solution space, the optimization procedure then seeks to find the optimum weights (w from (17)) as follows:

$${min}_{w = [ {w}_{1} … {w}_{N} ]} f ( {w}_{1} {A}_{1} + … + {w}_{N} {A}_{N} ) \qquad ( 23 )$$

The ability of this approach to find the true solution space relies on the fact that typically, the matrices Aₙ are distributed quite differently from realistic transmission matrices. For example, they might be non-sparse whereas real transmission matrices are typically very sparse. This means that random linear combinations of Aₙ will typically produce mostly non-zero elements in the transmission matrix estimate Aₑₛₜ (=w₁A₁+ . . . +w_{N}A_{N}). If the prior distribution favours sparsity then most combinations of weights will have a low likelihood. There is therefore a high probability that the most likely Aₑₛₜ is in fact the correct one. Whilst this explanation has not, at present, been rigorously proven to converge on the correct answer, it has the significant advantage that it only requires two reflectors (i.e. the reflector assembly 42 shown in FIG. 3A). This means a simpler design with less power loss.

A second approach seeks to achieve optimization through the addition of a third reflector 48. By recursive substitution of (21) and (22), the following expression can be written:

bₑₛₜ=B_{α}B_{α}⁺B_{β}B_{β}⁺ . . . B_{α}B_{α}⁺B_{β}B_{β}⁺bₑₛₜ

bₑₛₜ=(B_{α}B_{α}⁺B_{β}B_{β}⁺)_{M}bₑₛₜ (24)

where M is typically >3.

Equation (24) represents and adapted version of the power method for finding dominant eigenvalues of a matrix (described in E. Bodewig, Matrix Calculus. 1959, which is hereby incorporated by reference in its entirety) because the eigenvalue of the matrix (B_{α}B_{α}⁺B_{β}B_{β}⁺)^{M} corresponding to the desired solution is known to be real (=1) and is dominant as only a single solution exists (all other eigenvalues should tend to 0). By applying the power method with large M, any input, b′ₑₛₜ, to the right hand side of (24) will produce the desired eigenvector best when multiplied by the matrix (B_{α}B_{α}⁺B_{β}B_{β}⁺)^{M}. Alternatively, after only a small number of iterations, the dominant eigenvector of (B_{α}B_{α}⁺B_{β}B_{β}⁺)^{M} will be the desired solution and can be obtained by standard eigendecomposition of (B_{α}B_{α}⁺B_{β}B_{β}⁺)^{M}

This optimization approach has the advantage that no prior knowledge is required to produce a unique solution. Furthermore, it is not an iterative process and can recover transmission matrices in a single step. On the other hand, three reflectors 48 are required (i.e. utilizing the reflector assembly 42 shown in FIG. 4A) as opposed to the two (as shown in FIG. 3A) that are required for the prior distribution optimization approach described above.

Given the limitations of the zeroth-order approach, an alternative model is considered that relates TMs at different wavelengths, λₚ and λ_{q}, as:

$${A}_{{λ}_{q}} = {e}^{\frac{{λ}_{p}}{{λ}_{q}} log {A}_{{λ}_{p}}} \qquad ( 25 )$$

where log(A_{λ}_{ₚ}) is the matrix logarithm of A_{λ}_{ₚ}. This is derived using a coupled-mode theory treatment of optical fibres to model a linear change in effective optical path length of the fibre 26 as wavelength is varied. The full relationship between fibre TMs at different wavelengths is complex so the linear approximation is limited to the ‘spectral bandwidth’ of the fibre 26 in this instance. The model considers a length of MMF as a sequence of infinitesimal segments that introduce field coupling between modes, dm∈ [symbol] ^{2M×1}, such that:

dm=dAm (26)

where dA∈ [symbol] ^{2M×2M} represents an infinitesimal coupling matrix, and m∈ [symbol] ^{2M×1} represents the field in each of M spatial modes over 2 orthogonal polarisations. This matrix equation represents a system of 2M linear differential equations. The solution to these equations for any arbitrary input set of modes, x, after travelling a distance [symbol] along the central axis of the fibre 26 is given by a matrix exponential:

y′= [symbol] (27)

Since the TM A_{λ1}= [symbol] where [symbol] _{λ}_{₁} is the optical path length along the central fibre axis at wavelength λ₁, we can obtain dA from the matrix logarithm of A_{λ}_{₁} by:

$$d A = \frac{log {A}_{{λ}_{1}}}{{ℓ}_{{λ}_{1}}} \qquad ( 28 )$$

Matrix logarithms in general produce degenerate solutions with eigenvalues of the form γ+i2πn/ [symbol] _{λ}_{₁}, n∈ [symbol] but because of the bandwidth-limitation imposed here only n=0 needs to be considered. Neglecting dispersion within the relatively small (<10 nm) spectral bandwidth, a reasonable assumption for typical glasses, the equivalent optical path length, [symbol] _{λ}_{_{q}}, at the other characterisation wavelengths (λ_{q}=λ₂ . . . λ_{Q}) is determined:

$${ℓ}_{{λ}_{Q}} = \frac{{λ}_{1}}{{λ}_{q}} {ℓ}_{{λ}_{1}} \qquad ( 29 )$$

Substituting Equations 28 and 29 into Equation 27 produces Equation 25 as desired.

In contrast to the zeroth-order model, when the TMs are related by this first-order model it is not straightforward to solve for A_{λq} based on Equation 2 applied at different wavelengths. Therefore, an optimisation-based approach is presented that can compute A_{λ}_{_{q}} for λ_{q}=λ₁, . . . , λ_{Q} by repeatedly solving Equation 2 at different wavelengths, exploiting the relationship given by Equation 25. This approach, summarised in Algorithm 1, takes as input R_{λ}_{_{q}} with λ_{q}=λ₁, . . . , λ_{Q}λ, which are measured in advance, and C_{λ}_{_{q}} with λ_{q}=λ₁, . . . λ_{Q}, which are measured in situ.

Algorithm 1 Recovering TMs using first-order model

Input: R_{λ}_{_{q}}, C_{λ}_{_{q}}, λ_{q}, q = 1 . . . Q

| | |
|---|---|
| 1: | Solve zeroth-order model to obtain initial estimate of TM at wave- |
| | length λ₁, Â_{λ}_{₁} |
| 2: | while convergence criteria not met do |
| 3: | for λ_{q} = λ₁ . . . λ_{Q-1} do |
| 4: | Starting from Â_{λ}_{_{q}}, estimate TM at wavelength λ_{q}: Â_{λ}_{_{q}}_{′} = arg |
| | min_{A} \|\|A^{T}R_{λ}_{_{q}}A − C_{λ}_{_{q}}\|\| |
| 5: | Convert TM estimate to wavelength ${λ}_{q + 1} \text{:} {\hat{A}}_{{λ}_{q + 1}} = {e}^{\frac{{λ}_{q}}{{λ}_{q + 1}} log ( {\hat{A}}_{{λ}_{q}}^{′} )}$ |
| 6: | end for |
| 7: | Starting from Â_{λ}_{_{Q-1}}, estimate TM at wavelength λ_{Q}: Â_{λ}_{_{Q}}_{′} = arg |
| | min_{A} \|\|A^{T}R_{λ}_{_{Q}}A − C_{λ}_{_{Q}}\|\| |
| 8: | Convert TM estimate to wavelength ${λ}_{1} \text{:} {\hat{A}}_{{λ}_{1}} = {e}^{\frac{{λ}_{Q}}{{λ}_{1}} log ( {\hat{A}}_{{λ}_{Q}}^{′} )}$ |
| 9: | Compute convergence criteria |

10: end while
Output: TM at wavelength $λ \text{:} {\hat{A}}_{λ} = {e}^{\frac{{λ}_{1}}{λ} log ( {\hat{A}}_{{λ}_{1}}^{′} )}$

A solution to each sub-optimization problem within the loop, [symbol] _{λ}_{_{q}}, may be found using an iterative gradient descent solver but will not be unique because of the quadratic form of Equation 2. By converting between wavelengths in the iterative process above, such local minima may be avoided as they are typically not minima at other wavelengths, allowing the global minimum to be found (save for a global sign error). The convergence criteria may be defined as the number of iterations, or an error metric such as the relative change in gradient between iterations. Both metrics are used in this work and for numerous simulated and experimentally measured TMs it is observed empirically that [symbol] _{λ}_{_{q}} reliably converges to the true value of A_{λ}_{_{q}} for λ_{q}=λ₁, . . . , λ_{Q}.

As will be appreciated, in certain embodiments of the invention, the projected calibration patterns are not initially separated into modes. Rather, all incident modes are reflected without any time delays being introduced. More specifically, a random superposition of all fibre modes (since each optical wavelength is distributed across a random superposition of all fibre modes) is reflected and arbitrarily recombined/mixed. Advantageously, this approach can utilize an arbitrary number of fibre modes thereby making it particularly suitable for imaging (which requires a large number of modes).

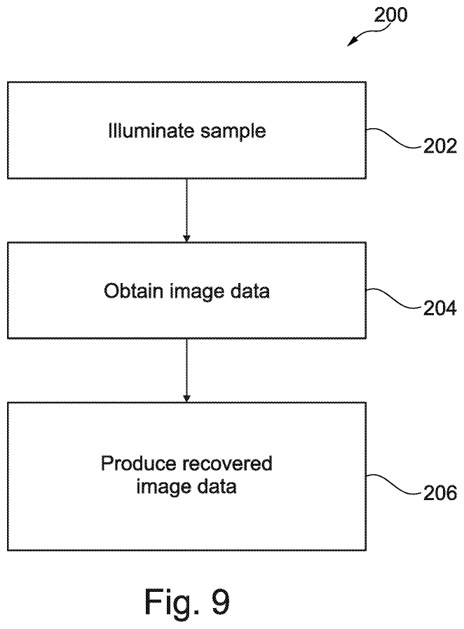

Once the instantaneous transmission matrix has been determined (e.g. by the above-described methods), the sample 30 may be imaged. FIG. 9 illustrates a method 200 of imaging according to an embodiment of the present invention. The method 200 comprises illuminating the sample 30, at step 202, where the sample 30 is proximate to the distal end 26b of the optical fibre 26 and the imaging wavelength is selected from the characterization wavelengths. Image data relating to the sample is obtained at the proximal end 26a of the optical fibre 26 at step 204. Using the obtained image data and the instantaneous transmission matrix, recovered image data is produced at step 206. In particular, the inverse of the instantaneous transmission matrix is used to produce the recovered image data from the obtained image data. The step of producing recovered image data may comprise producing recovered phase data, and/or producing recovered phase data, and/or producing recovered polarization data.

A non-limiting example of method 200 is set out below.

Imaging is performed using a new wavelength, the imaging wavelength λ_{Q+1}, which is longer than the wavelengths used for TM characterisation, i.e. λ_{Q+1}>λ₁, . . . , λ_{Q}, in order that light may pass through the reflector assembly 42. The TM at this wavelength, A_{λ}_{_{Q+1}}, is computed from the recovered A_{λ}_{₁} using Equation 25. Considering the physical model in the context of TM characterisation (FIG. 2D), a known illumination vector, xᵢₗₗᵤₘ, at wavelength λ_{Q+1} is first projected (step 202) onto the proximal facet 26a of the fibre 26, giving at the distal facet 26b:

y′ᵢₗₗᵤₘ=A_{λ}_{_{Q+1}}xᵢₗₗᵤₘ (30)

Next, the physical model from the perspective of imaging (FIG. 2E) is considered, where light passes through the full reflector assembly 42, which at wavelength λ_{Q+1} must be partially transmissive. Denoting the TM of the reflector assembly 42 at λ_{Q+1} by A_{refl}∈ [symbol] ^{2N×2M}, the illumination filed exiting the reflector stack is then:

y″ᵢₗₗᵤₘ=A_{refl}A_{λ}_{_{Q+1}}xᵢₗₗᵤₘ (31)

with y″ᵢₗₗᵤₘ∈ [symbol] ^{2N}, Setting N≥M allows oversampled illumination fields. y″ᵢₗₗᵤₘ can be propagated by some linear operator, G∈ [symbol] ^{2N×2N}, through free-space (e.g. Fresnel or Fraunhofer propagation) before reaching the target 30 where the field will be:

y″ᵢₗₗᵤₘ=GA_{refl}A_{λ}_{_{Q+1}}xᵢₗₗᵤₘ (32)

G is parameterised by the distance, d, between the sample 30 and the distal surface of the reflector assembly 42 (FIG. 2E). In general, d is not known a priori but can be estimated during operation.

Representing the target by a vector, r_{target}∈ [symbol] ^{2N}, an element-wise (Hadamard) product is performed with the illumination to give:

x′″_{target}=y′″ᵢₗₗᵤₘ·r_{target} (33)

This light reflected from the target 30 propagates backwards, first through free-space G^{T} then through the reflector assembly 42, A_{refl}^{T} to produce a field at the distal facet:

x′_{target}=A_{refl}^{T}G^{T}x′″_{target} (34)

Next, the illumination light that is reflected back from the reflector assembly 42 is also considered:

x′_{refl}=R_{λ}_{_{Q+1}}A_{λ}_{_{Q+1}}xᵢₗₗᵤₘ

We sum the two fields and propagate back through the fibre 26 giving:

yₜₒₜₐₗ=A_{λ}_{_{Q+1}}^{T}(x′_{target}+x′_{refl}) (35)

as the measured quantity at the camera plane, thus obtaining image data (step 204).

The goal is to recover r_{target} from the raw measured data, yₜₒₜₐₗ (Equation 35). To do this, x′″_{target} is first recovered by rearrangement of Equation 35 and substitution of Equations 34 and 33:

(A_{λ}_{_{Q+1}}^{T})⁻¹yₜₒₜₐₗ=(x′_{target}+x′_{refl})

x′_{target}=(A_{λ}_{_{Q+1}}^{T})⁻¹yₜₒₜₐₗ−R_{λ}_{_{Q+1}}A_{λ}_{_{Q+1}}xᵢₗₗᵤₘ

x′″_{target}=(A_{refl}^{T}G^{T})⁺((A_{λ}_{_{Q+1}}^{T})⁻¹yₜₒₜₐₗ−R_{λ}_{_{Q+1}}A_{λ}_{_{Q+1}}xᵢₗₗᵤₘ) (36)

where ( . . . )⁺ a generalised inverse because A_{refl} may be non-square. A_{refl} and R_{λ}_{_{Q+1}} are known from calibration prior to first use, A_{λ}_{_{Q+1}}, is estimated using Equation 25 and A_{λ}_{₁} (recovered using Algorithm 1), G can be estimated in postprocessing, xᵢₗₗᵤₘ is determined by the operator and yₜₒₜₐₗ is the measured returned field. Therefore all quantities on the right hand side (RHS) of Equation 36 are known, enabling recovery of x′″_{target}. An example of a full imaging process in accordance with an embodiment of the present invention is summarized in Algorithm 2. In alternative embodiments, the imaging wavelength may be less than each of the wavelengths used for TM characterisation or selected from the wavelengths used for TM characterisation.

Algorithm 2 Image recording and recovery

Input: xᵢₗₗᵤₘ, yₜₒₜₐₗ, G (estimated using d), σ, R_{λ}_{_{Q+1}}, A_{λ}_{_{Q+1}} and A_{refl}

| | |
|---|---|
| 1: | Estimate TM at imaging wavelength, A_{λ}_{_{Q+1}}, in situ using process of |
| | Section II C. |
| 2: | Send known illumination profile, xᵢₗₗᵤₘ into fibre. |
| 3: | Record returned field yₜₒₜₐₗ |
| 4: | Estimate G by estimating distance, d, between distal facet and sample. |
| 5: | Recover illuminated sample estimate: |

x_{target}_{^{′′′}}= (A_{refl}^{T}G^{T})⁺((A_{λQ+1}_{^{T}})⁻¹ yₜₒₜₐₗ − R_{λ}_{_{Q+1}}A_{λ}_{_{Q+1}}xᵢₗₗᵤₘ)

| | |
|---|---|
| 6: | Compute probability each sample pixel is produced by noise: |

${p}_{n} ( x , y ) = {χ}_{CDF}^{2} ( 1 , \frac{{‖ {x}_{target}^{′′′} ( x , y ) ‖}^{2}}{{σ}^{2}} )$
pₙ = [pₙ(x₁, y₁), pₙ(x₂, y₂), . . . ]

| | |
|---|---|
| 7: | Apply probability illumination correction to x_{target}^{′′′} |

\tilde{r}_{target} = xᵢₗₗᵤₘ_{^{′′′}} [symbol] (((1 − pₙ) ∘ |yᵢₗₗᵤₘ_{^{′′′}}| + pₙ) ∘ e^{iarg(y}^{ᵢₗₗᵤₘ}^{^{′′′}}⁾)

| | |
|---|---|
| 8: | if image appears out-of-focus then |
| 9: | adjust d then go to step 4 |

10: end if

11:

Output: Recovered image. \tilde{r}_{target}

Whilst the prior art methods have indirectly used phase and/or polarisation information to recover amplitude-only images from MMFs, embodiments of the present invention are capable of directly retrieving wide-field en-face images of these properties together with amplitude information.

Holographic endoscopes utilizing the optical system described above (and the associated methods) may record high resolution (e.g. 9.0±2.6 μm amplitude, phase; 36.0±10.4 μm polarimetric) images at working distances up to 1-2 cm and fields-of-view of ˜1×1 cm, in line with conventional endoscopy. The ability to adaptively change working distance without distal optics is a key advantage associated with embodiments of the present invention. Embodiments of the present invention offer potential ‘red-flag’ surveying of large areas for abnormalities, followed by zooming in to perform high resolution ‘optical biopsy’, which is currently impossible in a single device.

A further advantage associated with embodiments of the present invention is that is sensitive to a wider range of optical parameters relative to prior art devices and methods. In principle, this improved sensitivity is sufficient to observe small, localised proliferations of cells associated with early tumorigenesis.

Quantitative phase and resolved polarimetric properties have shown promise for enhancing cancer detection in ex vivo and in vivo rigid endoscope studies. Further, they are largely robust to variations in absolute intensity under varying measurement conditions. Retrieval of these properties through a flexible endoscope could therefore enhance contrast for pre-malignant and malignant changes during diagnostic endoscopy in a subject.

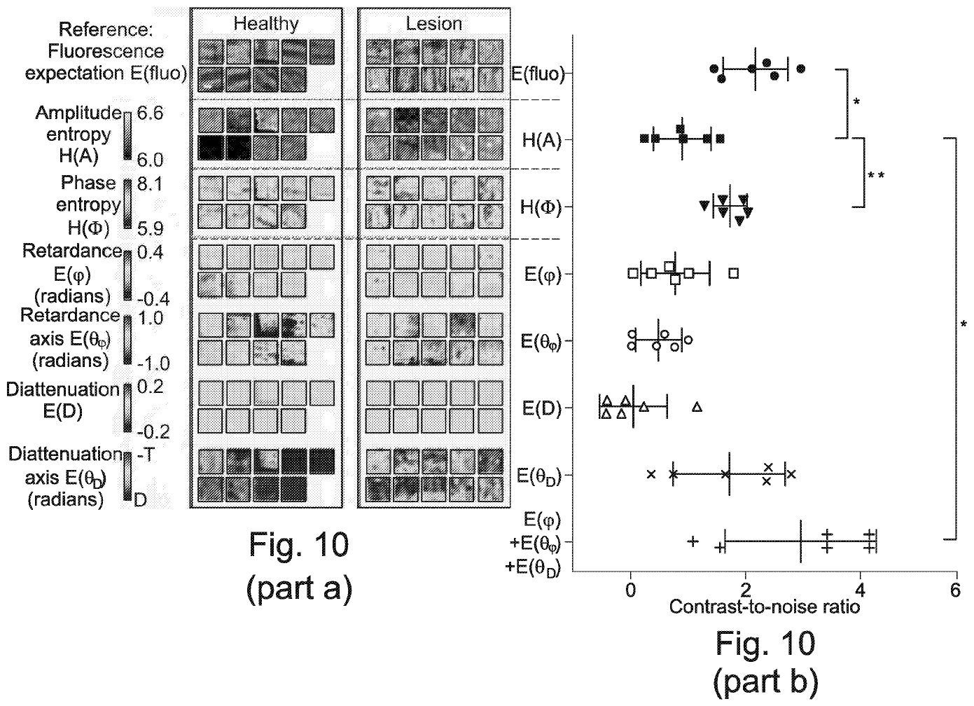

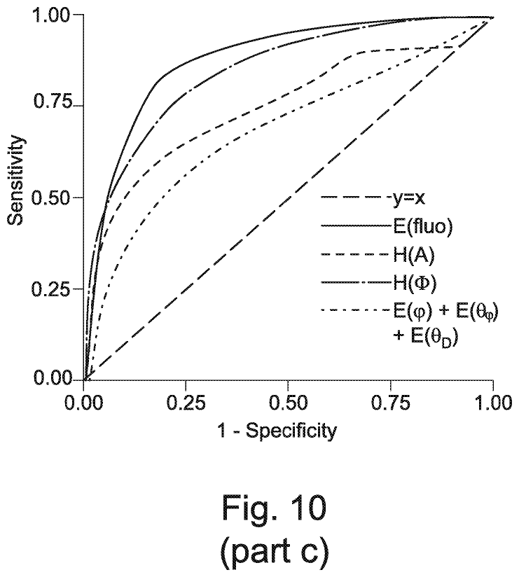

FIG. 10 part (a) shows a composite image that shows sections of healthy tissue and lesions from 9 samples (mouse oesophagus) and 5 endoscope modalities. A range of focal and diffuse early tumour lesions can be observed in the reference fluorescence images. Part (b) of FIG. 10 shows the contrast-to-noise ratio for the different modalities calculated independently for each of the 6 samples containing lesions. The phase entropy and sum φ, θ_{q}, and θ_{D} polarization parameters produce contrast statistically comparable to fluorescence imaging, which is considered to be the “gold standard” reference modality (p=0.148 and p=0.160 respectively) (scale bar 400 μm). Significance is determined by paired two-tailed t-test, * p<0/05, ** p<0.01. Part (c) of FIG. 10 shows receiver operating characteristic curves illustrating the performance of different modalities when a binary classifier with varying threshold is applied to discriminate between healthy and lesion tissue. Phase entropy significantly out-performs conventional amplitude imaging, but he large inter-sample variance of the combined polarization metric results in reduced performance with a simple binary threshold classifier.

Obtaining a label-free diagnosis equivalent to that provided by fluorescence staining avoids the need for extended procedure time and potential toxicities associated with in vivo dye application. Importantly, phase and polarimetric images may contain more relevant diagnostic information than amplitude-only images, demonstrating a key advance over what is possible using current commercial endoscopes. The ability to holographically produce illumination patterns at the distal end of the fibre, once the transmission matrix is known, would also enable other modalities of imaging such as fluorescence. It could even pave the way for super-resolution imaging techniques such as STED or STORM through an endoscope.

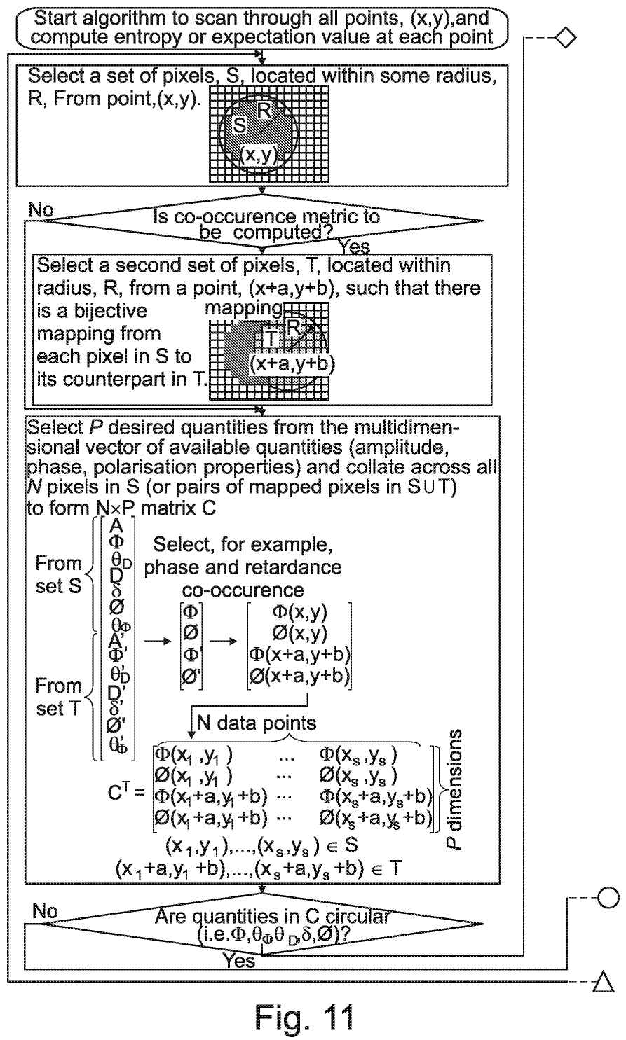

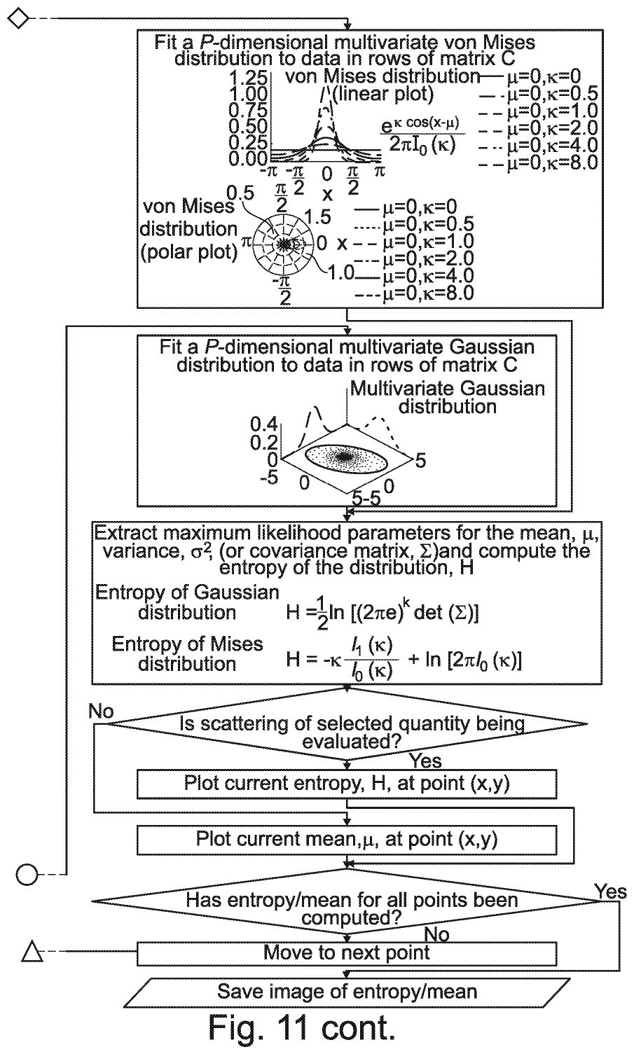

FIG. 11 shows a method of producing an image of entropy/mean from recovered image data in accordance with an embodiment of the present invention. Such images may be used to determine the presence of a physiological condition in a subject e.g. cancer or a pre-cancerous condition, the presence of scar tissue, or the presence of inflammation.

In accordance with an aspect of the present invention, there is provided a method of determining a presence of a physiological condition in a subject, the method comprising: producing recovered image data relating to a tissue sample using a method as described above or the optical system described above; and determining, in dependence on the recovered image data, a presence of the physiological condition in the tissue sample.

The step of determining a presence of the physiological condition in the tissue sample may comprise:

using the recovered image data as contrast mechanisms to identify tissue morphology or microstructure associated with the physiological condition; and/or

using the recovered image data to produce images showing the spatial variation or spatial entropy of one or more of amplitude, phase and polarisation, and using differences thereof to delineate healthy areas of the tissue sample (e.g. areas not affected by the physiological condition) and areas of the tissue sample that are affected by the physiological condition (e.g. as described above with reference to FIG. 11).

The physiological condition may, for example, be cancer or a pre-cancerous condition, the presence of scar tissue, or the presence of inflammation. The above described method may be useful in determining the presence of other physiological conditions.

In accordance with another aspect of the present invention, there is provided a computer implemented method comprising:

receiving recovered image data relating to a tissue sample obtained using a method as described above or the optical system described above; and

determining, in dependence on the recovered image data, a presence of a physiological condition in the tissue sample.

The step of determining a presence of the physiological condition in the tissue sample may comprise:

using the recovered image data as contrast mechanisms to identify tissue morphology or microstructure associated with the physiological condition; and/or

using the recovered image data to produce images showing the spatial variation or spatial entropy of one or more of amplitude, phase and polarisation, and using differences thereof to delineate healthy areas of the tissue sample (e.g. areas not affected by the physiological condition) and areas of the tissue sample that are affected by the physiological condition (e.g. as described above with reference to FIG. 11).

The physiological condition may, for example, be cancer or a pre-cancerous condition, the presence of scar tissue, or the presence of inflammation. The above described method may be useful in determining the presence of other physiological conditions.

Embodiments of the present invention may form part of or be utilized in an instrument that improves early detection of cancer, and thus improve survival rates (e.g. from oesophageal cancer).

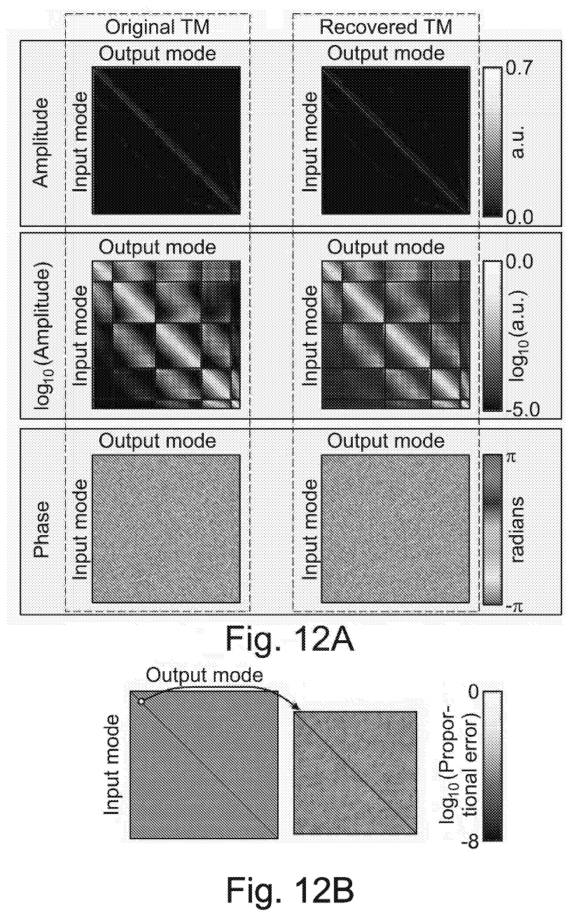

FIG. 12A shows an example of simulated recovery of an experimentally-measured 1648×1648 transmission matrix from a 2 m length of multicore optical fibre using a method according to an embodiment of the present invention. The simulated recovery involved the use of a physically realistic simulation of a reflector stack. Specifically, FIG. 12A shows the original transmission matrices and the corresponding first-order recovered transmission matrices (which show high visual similarity, thus indicating successful recovery). FIG. 12B shows the proportional element-wise error in the transmission matrix recovery of FIG. 12A (maximum <10⁻³).

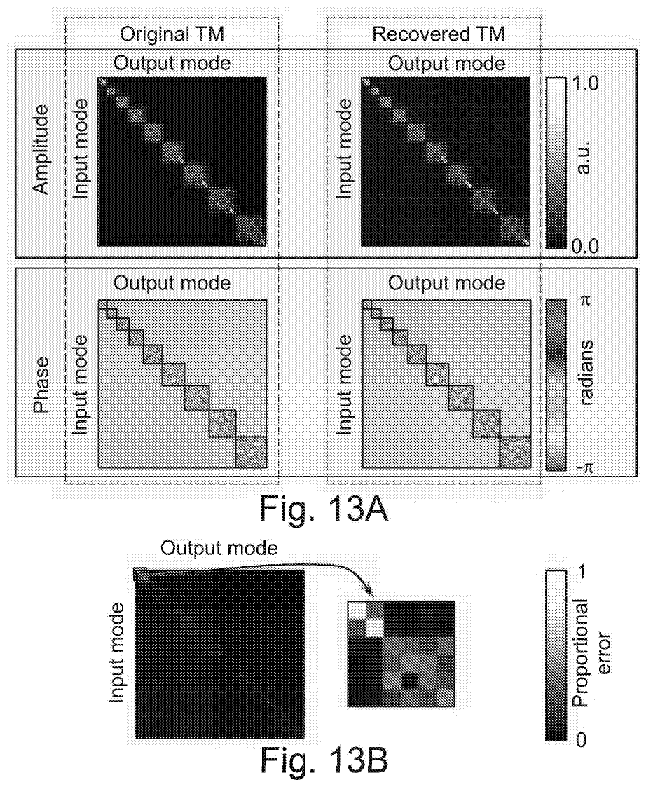

FIG. 13A shows an example of a simulated recovery of an experimentally-measured step-index multimode fibre, using transmission matrices recorded at multiple wavelengths (1525.6 nm, 1526.5 nm, 1527.4 nm and 1528.3 nm) and a simulated reflector stack. Specifically, FIG. 13A shows the original transmission matrices and the corresponding first-order recovered transmission matrices (which show high visual similarity, thus indicating successful recovery). FIG. 13B shows the proportional element-wise error in the transmission matrix recovery of FIG. 13A (typically <0.05 except for the very lowest order modes that exhibit error ˜0.2 due to phase error.

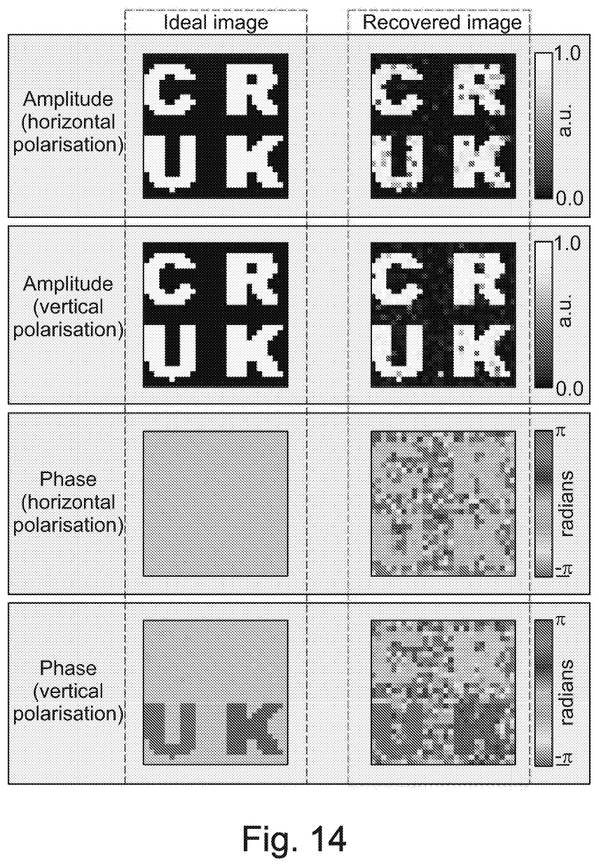

FIG. 14 shows an example of simulated reconstruction of an amplitude, phase and polarisation image of a target at the distal facet using an experimentally-measured transmission matrix. The recovered image error is largely driven by illumination correction.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of characterizing an optical system, wherein the optical system comprises an optical fibre having a proximal end and a distal end, wherein the optical fibre comprises non-single-mode optical fibre, and a reflector assembly comprising a stack of reflectors disposed at the distal end of the optical fibre, wherein the stack of reflectors is arranged to provide different reflector matrices in dependence on illumination wavelength, the method comprising:
projecting calibration patterns at a plurality of characterization wavelengths onto the proximal end;
obtaining, at the proximal end, data relating to reflected calibration patterns; and
determining an instantaneous transmission matrix of the optical fibre using the data relating to the reflected calibration patterns and the reflector matrices.

2. The method of claim 1, wherein the reflector matrices are determined by:
determining a characterization transmission matrix of the optical fibre in a characterization configuration;
determining, at each of the plurality of characterization wavelengths, a reflectance matrix of the optical fibre in the characterization configuration with the stack of reflectors disposed at the distal end; and
determining the reflector matrices using the reflectance matrices and the characterization transmission matrix.

3. The method of claim 2, wherein determining the characterization transmission matrix comprises:
transmitting light through the optical fibre in the characterization configuration at each of the plurality of characterization wavelengths;
detecting the transmitted light at each of the plurality of characterization wavelengths; and
determining the characterization transmission matrix using the detected transmitted light.

4. The method of claim 1, wherein detecting reflected calibration patterns comprises measuring one or more of (i) the amplitude of reflected calibration patterns, (ii) the phase of reflected calibration patterns, and (iii) the polarization of reflected calibration patterns.

5-6. (canceled)

7. The method of claim 1, comprising producing a square reflectance matrix from the reflected calibration patterns, wherein determining the instantaneous transmission matrix comprises using the square reflectance matrix.

8. A method of imaging, the method comprising:
characterizing the optical system claim 1;
illuminating a sample proximate to the distal end at an imaging wavelength;
obtaining, at the proximal end, image data relating to the sample; and
producing recovered image data using the image data relating to the sample and the instantaneous transmission matrix.

9. The method of claim 8, further comprising obtaining a transmission matrix of the reflector assembly, wherein determining the instantaneous transmission matrix or producing recovered image data comprises using the transmission matrix of the reflector assembly.

10. The method of claim 8, wherein producing recovered image data comprises producing one or more of (i) recovered amplitude data, (ii) recovered phase data, and (iii) recovered polarization data.

11-12. (canceled)

13. The method of claim 8, wherein the sample comprises human or animal tissue.

14. The method of claim 8, wherein the sample is in vivo, ex vivo, or in vitro.

15. The method of claim 8, wherein the imaging wavelength is one of (i) selected from the characterization wavelengths, (ii) greater than each of the characterization wavelengths, and (iii) less than each of the characterization wavelengths.

16. (canceled)

17. An optical system comprising:
an optical fibre having a proximal end and a distal ends; and
a reflector assembly comprising a stack of reflectors disposed at the distal end of the optical fibre, wherein the stack of reflectors is arranged to provide different reflector matrices in dependence on illumination wavelength.

18. The optical system of claim 17, wherein the stack of reflectors comprises a plurality of reflectors.

19. The optical system of claim 18, wherein each of the plurality of reflectors is separated from an adjacent reflector by an absorptive filter.

20. The optical system of claim 19, wherein the plurality of reflectors comprises optical metasurfaces.

21. The optical system of claim 17, further comprising:
an illumination source optically coupled to the optical fibre;
means for generating a calibration pattern for projection onto the proximal end of the optical fibre; and
detection means for detecting images from the proximal end of the optical fibre.

22. A method of determining a presence of a physiological condition in a subject, the method comprising:
producing recovered image data relating to a tissue sample using the method of claim 8; and
determining, in dependence on the recovered image data, a presence of the physiological condition in the tissue sample.

23. The method of claim 22, wherein the step of determining a physiological condition in the tissue sample comprises at least one of:
using the recovered image data as contrast mechanisms to identify tissue morphology or microstructure associated with the physiological condition; and
using the recovered image data to produce images showing the spatial variation or spatial entropy of one or more of amplitude, phase, and polarization, and using differences thereof to delineate healthy areas of the tissue sample and areas of the tissue sample that are affected by the physiological condition.

24. The method of claim 22, wherein the physiological condition is cancer or a pre-cancerous condition.

25. A computer implemented method comprising:
receiving recovered image data relating to a tissue sample obtained using a method of claim 8; and
determining, in dependence on the recovered image data, a presence of a physiological condition in the tissue sample.

26. The computer implemented method of claim 25, wherein the step of determining the physiological condition in the tissue sample comprises at least one of:
using the recovered image data as contrast mechanisms to identify tissue morphology or microstructure associated with the physiological condition; and
using the recovered image data to produce images showing the spatial variation or spatial entropy of one or more of amplitude, phase, and polarization, and using differences thereof to delineate healthy areas of the tissue sample and areas of the tissue sample that are affected by the physiological condition.
